(19) United States
(12) Patent Application Publication
(10) Pub. No.: US 2017/0135675 A1
(43) Pub. Date: May 18, 2017

(54) ADAPTIVE CLUTTER DEMODULATION FOR ULTRASOUND IMAGING

(71) Applicant: VANDERBILT UNIVERSITY, Nashville, TN (US)

(72) Inventors: Jaime Tierney, Nashville, TN (US); Brett Byram, Brentwood, TN (US); Douglas Dumont, Washington, DC (US)

(21) Appl. No.: 15/351,123

(22) Filed: Nov. 14, 2016

Related U.S. Application Data
(60) Provisional application No. 62/254,290, filed on Nov. 12, 2015.

(51) Int. Cl. A61B 8/08; A61B 8/06; A61B 8/00

(52) U.S. Cl. CPC A61B 8/5276; A61B 8/5207; A61B 8/4483; A61B 8/461; A61B 8/488; A61B 8/06

(57) ABSTRACT

Systems and methods for adaptive clutter demodulation in ultrasound imaging are provided. The method includes obtaining a plurality of beamformed radiofrequency (RF) lines representing an ultrasound scan and computing displacements between adjacent ones of the plurality of beamformed RF lines. The method also includes generating shifted RF lines through depth based on the computed displacements and normalizing the shifted RF lines to yield normalized RF lines. The method also includes assembling an ultrasound image based on the normalized RF lines.

### CROSS-REFERENCE TO RELATED APPLICATIONS

This application claims priority to and the benefit of U.S. Provisional Patent Application No. 62/254,290, entitled “Adaptive clutter demodulation scheme for measuring low velocity blood flow,” filed on Nov. 12, 2015, the contents of which are herein incorporated by reference in their entirety.

### STATEMENT REGARDING FEDERALLY FUNDED RESEARCH

This invention was made with government support under grant numbers 1RO1HL128715 and S10OD016216-01 awarded by National Institutes of Health. The government has certain rights in the invention

### FIELD OF THE INVENTION

The present invention relates to ultrasound imaging, and more specifically to apparatus and methods for adaptive clutter demodulation in ultrasound imaging.

### BACKGROUND

Blood perfusion imaging with ultrasound is still extremely challenging but also clinically invaluable. Perfusion, which encompasses the slowest flow in the smallest vasculature, is a crucial component for evaluating applications like tumor treatment and monitoring because it involves the exchange of nutrients between blood and tissue. Measuring perfusion with ultrasound is challenging because tissue motion artifacts, otherwise known as tissue clutter, interfere with the signal from slowly moving blood. Contrast agents have been employed to enhance ultrasound imaging for measuring slower flow [1]. However, apart from adding to the cost, invasiveness, and variability of tissue clutter interference problem, ultrasound imaging without contrast agent could enable unlimited real-time measurements of perfusion, which would substantially improve current understanding, monitoring, and diagnosis of vascular diseases and cancers.

The signal from tissue has been variably reported in the literature as being as low as 10-100 times and up to 100 dB larger than the signal from blood [2-7]. Filters are therefore required in conventional ultrasound blood flow imaging to remove the overpowering tissue clutter signal and uncover the signal from blood flow. Apart from the tissue signal being stronger than the blood signal, tissue also moves at lower frequencies or velocities than most blood flow. Therefore, conventional techniques that use standard high pass filters are sensitive to blood motion that is faster than tissue motion. For lower blood velocities, conventional high pass filters become less effective because tissue clutter can have Doppler frequencies similar to or even higher than those from slower flow. This results in the tissue clutter signal overlapping with, and overpowering, the signal from lower velocity blood flow. Conventional Doppler methods are therefore limited to blood velocities higher than those of tissue clutter. Two primary sources of tissue clutter are sonographer hand motion and patient physiological motion. Considering only these sources of clutter, Heimdal et al. estimated theoretical lower bounds on blood velocity estimates at different imaging frequencies with conventional Doppler imaging [4]. Based on this work, it has been long claimed that ultrasound without contrast agent is limited to blood velocities greater than 5 mm/s for center frequencies less than 8 MHz [1, 4]. This limitation eliminates sensitivity to small vessels such as capillaries, venules, and 17-32 μm diameter arterioles, which have average velocities of 0.33 mm/s, 3.3 mm/s and 2-4 mm/s, respectively [4, 8]. Perfusion imaging is therefore seemingly impossible with conventional ultrasound methods because perfusion constitutes the slowest blood flow in the capillaries or smallest vasculature.

Conventional high pass filters have been investigated extensively for the purpose of suppressing tissue clutter in Doppler ultrasound imaging. Among the most commonly used are infinite and finite impulse response (IIR and FIR) filters and polynomial regression filters [5-7]. Each of these can be optimized in terms of frequency response parameters for maximal tissue removal and blood flow preservation. Additionally, since short ensemble lengths have made high pass filtering difficult in the past, the development of high frame rate acquisition techniques, including plane wave and plane wave synthetic aperture imaging, have allowed for the implementation of more effective high pass filtering [9, 10]. However, none of these conventional techniques account for the overlap between the tissue clutter and low velocity blood flow signals. Thus, even a perfectly optimized and sufficiently sampled conventional high pass filter will be unable to preserve slow flow.

Advanced pre-filtering techniques have been proposed to improve signal separation of tissue and slow flow. Among these is the method by Thomas and Hall, expanded upon by Bjaerum and Torp, which involves adaptively modulating the tissue clutter bandwidth to be centered around DC before applying a conventional high pass filter [3, 11]. This method is useful for conventional Doppler acquisitions with shorter ensemble lengths that can result in a non-zero tissue clutter mean Doppler frequency that interferes with uniform blood flow. However, with the development of the aforementioned high frame rate acquisition techniques, longer ensemble lengths are more easily achieved. With these longer acquisition sequences, the Doppler frequency shift of tissue clutter becomes less of a problem since tissue does not typically move uniformly over long periods of time and will therefore already be centered about DC. Furthermore, due to the spectral broadening of the tissue clutter signal caused by sonographer hand motion and patient physiological motion, even if the tissue clutter signal is modulated to be centered about DC, the bandwidth of the clutter still makes it impossible to separate the blood signal from the tissue clutter signal. Due to the tissue spectral broadening problem encountered in the frequency domain of the slow-time dimension (i.e., along the ensemble), other methods have been proposed that aim to incorporate time domain and/or spatial dimensional information to better separate tissue clutter from low velocity blood flow [7, 12]. These methods use singular value decomposition (SVD) or principle/independent component analysis (PCA/ICA) to take advantage of the temporally and spatially coherent nature of tissue compared to the temporally and spatially incoherent nature of blood flow. For example, Gallippi and Trahey proposed a time-domain blind source separation (BSS) technique that used principle or independent component analysis with polynomial regression to adaptively filter out tissue clutter [12]. Demene et al. also proposed an SVD algorithm using plane wave synthetic aperture imaging to incorporate the spatial characteristics of blood and tissue while also benefiting from large slow-time ensemble sizes [7]. Although these methods have improved slow flow measurements with ultrasound, the spectral broadening of the tissue clutter bandwidth is still an issue that ultimately limits the preservation of slow flow signal with the previously proposed methods.

Alternative beamforming methods have also been shown to improve slow flow estimation, including coherent flow power Doppler (CFPD) [13, 14]. CFPD increases sensitivity to slow flow by suppressing thermal noise and clutter [14]. However, spectral broadening of the tissue clutter signal will still limit estimation of the slowest flow.

### SUMMARY

Embodiments of the invention concern systems and methods for adaptive clutter demodulation in ultrasound imaging. In a first embodiment of the invention, there is provided a method. The method includes obtaining a plurality of beamformed radiofrequency (RF) lines representing an ultrasound scan and computing displacements between temporally adjacent ones of the plurality of beamformed RF lines. The method also includes generating shifted RF lines through depth based on the computed displacements and normalizing the shifted RF lines to yield normalized RF lines. The method also includes assembling an ultrasound image based on the normalized RF lines.

In the method, the computing can include calculating relative displacements between the plurality of beamformed RF lines and reconstructing absolute displacements for the plurality of beamformed RF lines based on the relative displacements. The relative displacements can be calculated using an autocorrelation method and the absolute displacements can be reconstructed by one of a modeling approach (e.g., a least squares approach) or an external motion tracking approach.

In the method, the generating of the shifted lines can include shifting from the computed absolute displacement to zero displacement. The generating of the shifted lines can be performed by calculating a non-rigid shift of the plurality of beamformed RF lines from the computed absolute displacement to zero displacement. This calculation can be performed using a shape-preserving piecewise cubic interpolation or other interpolation technique.

In the method, the normalizing can include scaling the shifted RF lines according to a ratio of a power of an envelope of the plurality of beamformed RF lines and an amplitude of the envelope of the plurality of beamformed RF lines. The normalizing further can also include, prior to the scaling, resampling the normalization function to account for additional non-rigid shifts in the shifted RF lines. For example, by median filtering the ratio through slow-time.

In a second embodiment, an ultrasound system is provided. The system includes at least one transducer front end, at least one display processor configured for assembling an ultrasound image based on received RF line data, and at least one Doppler processor coupled to the at least one transducer front end and the at least one display processor. In the system, the at least one Doppler processor is configured for obtaining, from the at least one transducer front end, a plurality of beamformed RF lines associated with an ultrasound scan and computing displacements between adjacent ones of the plurality of beamformed RF lines. The at least one Doppler processor is also configured for generating shifted RF lines through depth based on the computed displacements, normalizing the shifted RF lines to yield normalized RF lines, and transmitting data representing the normalized RF lines to the at least one display processor.

In the system, the computing can include calculating relative displacements between the plurality of beamformed RF lines and reconstructing absolute displacements for the plurality of beamformed RF lines based on the relative displacements. The relative displacements can be calculated using an autocorrelation method and the absolute displacements can be reconstructed by one of a modeling approach (e.g., a least squares approach) or an external motion tracking approach.

In the system, generating of the shifted lines can include shifting from the computed absolute displacement to zero displacement. The generating of the shifted lines can be performed by calculating a non-rigid shift of the plurality of beamformed RF lines from the computed absolute displacement to zero displacement. This calculation can be performed using a shape-preserving piecewise cubic interpolation or other interpolation technique.

In the system, the normalizing can include scaling the shifted RF lines according to a ratio of a power of an envelope of the plurality of beamformed RF lines and an amplitude of the envelope of the plurality of beamformed RF lines. The normalizing further can also include, prior to the scaling, resampling the normalization function to account for non-rigid shifts in the shifted RF lines. For example, by median filtering the ratio through slow-time.

In a third embodiment, there is provide a non-transitory computer-readable medium, having stored thereon a computer program executable by a computing device. The computer program includes code sections for causing the computing device to obtain a plurality of beamformed radiofrequency (RF) lines representing an ultrasound scan and compute displacements between adjacent ones of the plurality of beamformed RF lines. The computer program also includes code sections for causing the computing device to generate shifted RF lines through depth based on the computed displacements, normalize the shifted RF lines to yield normalized RF lines, and assemble an ultrasound image based on the normalized RF lines.

The computer-readable medium can also include code sections for causing the computing device to compute the displacements by calculating relative displacements between the plurality of beamformed RF lines and reconstruct absolute displacements for the plurality of beamformed RF lines based on the relative displacements.

The computer-readable medium can also include code sections for causing the computing device to generate the shifted lines by shifting the beamformed RF lines from the computed absolute displacement to zero displacement.

The computer-readable medium can also include code sections for causing the computing device to normalize the shifted RF lines by scaling the shifted RF lines according to a ratio of a power of an envelope of the plurality of beamformed RF lines and an amplitude of the envelope of the plurality of beamformed RF lines.

### BRIEF DESCRIPTION OF THE DRAWINGS

FIG. 1 is a flowchart of steps in an exemplary process in accordance with an exemplary embodiment;

FIG. 2a is an x-y plot of median bandwidths and corresponding velocities computed across depths and averaged across subjects for three beamforming methods before and after adaptive demodulation in accordance with the various embodiments;

FIG. 2b is an x-y plot of median bandwidths and corresponding velocities computed across depths and averaged across subjects are shown for a single plane wave beamforming method before and after adaptive demodulation in accordance with the various embodiments;

FIG. 2c is an x-y plot of median bandwidths and corresponding velocities computed across depths and averaged across subjects for an SPW acquisition method and for different steps of the adaptive demodulation scheme in accordance with the various embodiments;

FIGS. 3a and 3b are x-y plots of amplitude through slow-time for two example depths;

FIG. 4a is an x-y plot of median relative power with respect to the last in vivo arterial occlusion time point is plotted for every 50 ms for various filtering cases, including a filtering case in accordance with the various embodiments;

FIG. 4b shows power Doppler and corresponding B-mode images for 2, 8, 22, and 30 second time points of the in vivo arterial occlusion scan for each filtering case in FIG. 4a;

FIGS. 5a and 5b show power Doppler images for the 2 and 5 second time points of in vivo arterial occlusion scans and muscle contraction scans, respectively.

FIG. 6a is an x-y plot of median relative power with respect to the last in vivo muscle contraction time point plotted for every 50 ms for various filtering cases, including a filtering case in accordance with the various embodiments;

FIG. 6b shows power Doppler and corresponding B-mode images for 5, 17, 26, and 30 second time points of the in vivo muscle contraction scan for each filtering case in FIG. 6a; and

FIG. 7 shows an exemplary ultrasound device that can be configured for implementing the various embodiments.

### DETAILED DESCRIPTION

The present invention is described with reference to the attached figures, wherein like reference numerals are used throughout the figures to designate similar or equivalent elements. The figures are not drawn to scale and they are provided merely to illustrate the instant invention. Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. In other instances, well-known structures or operations are not shown in detail to avoid obscuring the invention. The present invention is not limited by the illustrated ordering of acts or events, as some acts may occur in different orders and/or concurrently with other acts or events. Furthermore, not all illustrated acts or events are required to implement a methodology in accordance with the present invention.

As noted above, due to spectral broadening caused by patient motion and sonographer motion, conventional Doppler methods are limited to blood flow above 5-10 mm/s for clinical imaging frequencies. In turn, this eliminates sensitivity to slow flow or perfusion [4]. To address this, the various embodiments are directed to an adaptive clutter demodulation scheme that suppresses the bandwidth of tissue clutter while still preserving signal from slow blood flow. This approach successfully reduces hand motion spectrum bandwidths, allowing for the detection of blood velocities well below assumed theoretical limits. Additionally, this type of filter results in a higher dynamic range between the lowest and highest blood flow time points compared to conventional filters for both in vivo studies.

Theory

Tissue Clutter and Blood Flow Models

A model for tissue vibration and blood flow has been previously derived by Heimdal and Torp [4]. For the purposes of describing the various embodiments, one can consider a simple realization of their classic model relevant to a single resolution cell. Assuming only stationary tissue is present in the field of view, the resulting Doppler signal at a given spatial location and slow-time point, t, could be represented as the sum of the complex amplitudes of the tissue scatterers,

$${s}_{tissue} ( t ) = {\sum }_{m = 0}^{M - 1} {A}_{m} \qquad ( 1 )$$

where Aₘ is the complex amplitude of a single scatterer and M is the total number of tissue scatterers. Since the scatterers are stationary over time, this signal is constant in the time domain and thus a delta function at DC in the frequency domain.

Similarly, if only blood were present, the resulting signal at time t could be represented as the sum of complex amplitudes modulated by the velocity term of each scatterer since each blood scatterer is moving at some variable speed,

$${s}_{blood} ( t ) = {\sum }_{n = 0}^{N - 1} {A}_{n} {e}^{j {ω}_{n} ( t ) t} \\ where \\ {A}_{n} \\ and \\ {ω}_{n} ( t ) = \frac{2 {v}_{n} ( t ) cos ( {θ}_{n} ) {ω}_{0} T}{c} \qquad ( 2 )$$

are the amplitude and angular frequency of a single blood scatterer, respectively, and N is the total number of blood scatterers. In the angular frequency equation, c is the speed of sound, vₙ(t) is the velocity of a single scatterer at time t, θₙ is the beam-to-flow angle of a single scatterer, ωₒ is the transmit frequency, and T is the time between pulses or the inverse of the pulse repetition frequency (PRF). Since blood scatterers will be moving at some distribution of velocities, this signal would be broad-band and centered about the mean frequency or velocity of the blood scatterers in the frequency domain.

When both stationary tissue and flowing blood are present simultaneously, the signals in (1) and (2) are summed, as

$${s}_{tissue + blood} ( t ) = {\sum }_{m = 0}^{M - 1} {A}_{m} + {\sum }_{n = 0}^{N - 1} {A}_{n} {e}^{j {ω}_{n} ( t ) t} \qquad ( 3 )$$

Conceptually, for this case, the tissue and flowing blood signals are well separated in the frequency domain. Tissue clutter is therefore easily removed with conventional techniques.

When sonographer hand motion and patient physiological motion are present, the signal will include an additional velocity term that describes the resulting axial motion of both the tissue and blood scatterers,

$${s}_{tissue + blood} ( t ) = ( {\sum }_{m = 0}^{M - 1} {A}_{m} + {\sum }_{n = 0}^{N - 1} {A}_{n} {e}^{j {ω}_{n} ( t ) t} ) \times {e}^{j {ω}_{physio + sono} ( t ) t} \qquad ( 4 )$$

where φ_{physio+sono} is the angular frequency produced by patient physiological and sonographer hand motion. This motion causes a phase modulation that contributes to the spectral broadening of the tissue clutter bandwidth and causes an overlap between the tissue clutter and blood flow signals in the frequency domain. This spectral broadening makes conventional high pass filtering of the tissue clutter signal difficult when trying to image lower velocity blood flow.

Adaptive Clutter Demodulation Model

The various embodiments are directed to a method that aims to estimate and correct for the patient physiological and sonographer hand motion in order to remove the added velocity term in (4),

$${s}_{tissue + blood} ( t ) = \begin{matrix}( {\sum }_{m = 0}^{M - 1} {A}_{m} + {\sum }_{n = 0}^{N - 1} {A}_{n} {e}^{j {ω}_{n} ( t ) t} ) \times \\ {e}^{j {ω}_{physio + sono} ( t ) t} \times {e}^{- j {\hat{ω}}_{physio + sono} ( t ) t}\end{matrix} \qquad ( 5 )$$

where \hat{ω}_{physio+sono} is an estimate of the angular frequency produced by patient physiological and sonographer hand motion. By correcting for this motion at each depth through slow-time, we are adaptively demodulating the tissue clutter bandwidth. In doing so, we are ideally left with (3), which, again, can easily be addressed with conventional filters.

However, due to both tissue motion and inherent scanner variability, there will also be an amplitude modulation that will further contribute to the spectral broadening of the tissue clutter signal [4]. Amplitude modulation from tissue motion could result from residual axial motion as well as lateral and elevational motion. Looking only at the tissue signal portion of (3) (i.e., (1)), after the described phase demodulation, this amplitude modulation can be simply described by a time dependence of the amplitude term in (1),

$${s}_{tissue} ( t ) = {\sum }_{m = 0}^{M - 1} {A}_{m} ( t ) \qquad ( 6 )$$

To correct for this time dependence of the tissue amplitude, the signal at each time point can be normalized to the amplitude of the envelope or magnitude of the signal at that time point. Additionally, to preserve the power of the original signal, each term can then be multiplied by the power of the envelope of the signal. These operations are summarized in the following equation,

$${f}_{norm} ( t ) = \frac{\sqrt{\frac{{\sum }_{l = 1}^{L} {\vert {s}_{tissue} ( l ) \vert}^{2}}{L}}}{\vert {\sum }_{m = 0}^{M - 1} {A}_{m} ( t ) \vert} \qquad ( 7 )$$

where L is the total number of slow-time points. By applying this correction to the tissue only signal, we remove the time dependence and are ideally left with (1),

$$\begin{matrix}{s}_{tissue} ( t ) = ( {\sum }_{m = 0}^{M - 1} {A}_{m} ( t ) ) \times {f}_{norm} ( t ) \\ = {\sum }_{n = 0}^{M - 1} {A}_{m}\end{matrix} \qquad ( 8 )$$

However, this operation becomes more complicated when applied to (3) since fₙₒᵣₘ(t) will reflect amplitude modulation of both tissue and blood and will subsequently also demodulate the blood signal amplitude (which will likely also exhibit a time dependence of the amplitude term),

$$\begin{matrix}{f}_{norm} ( t ) = \frac{\sqrt{\frac{{\sum }_{l = 1}^{L} {\vert {s}_{tissue + blood} ( l ) \vert}^{2}}{L}}}{\vert {\sum }_{m = 0}^{M - 1} {A}_{m} ( t ) + {\sum }_{n = 0}^{N - 1} {A}_{n} ( t ) {e}^{j {ω}_{n} ( t ) t} \vert} & ( 9 ) \\ {s}_{tissue + blood} ( t ) = ( {\sum }_{m = 0}^{M - 1} {A}_{m} ( t ) + {\sum }_{n = 0}^{N - 1} {A}_{n} ( t ) {e}^{j {ω}_{n} ( t ) t} ) {f}_{norm} ( t ) & ( 10 )\end{matrix}$$

To avoid demodulating the blood signal amplitude, we can take advantage of the difference in temporal coherence length between tissue and blood. The blood signal has a shorter coherence length so it is possible to apply a median filter to fₙₒᵣₘ(t) that is large enough to not incorporate changes in blood amplitude while still small enough to capture significant tissue amplitude modulation. In doing so, we can approximate (7) from (9),

$$\begin{matrix}{f}_{norm} ( t ) = R \{ \frac{\sqrt{\frac{{\sum }_{l = 1}^{L} {\vert {s}_{tissue + blood} ( l ) \vert}^{2}}{L}}}{\vert {\sum }_{m = 0}^{M - 1} {A}_{m} ( t ) + {\sum }_{n = 0}^{N - 1} {A}_{n} ( t ) {e}^{j {ω}_{n} ( t ) t} \vert} , k \} \\ \approx \frac{\sqrt{\frac{{\sum }_{l = 1}^{L} {\vert {s}_{tissue} ( l ) \vert}^{2}}{L}}}{\vert {\sum }_{m = 0}^{M - 1} {A}_{m} ( t ) \vert}\end{matrix} \qquad ( 11 )$$

where R{x,k} represents the median filter operation on signal x of size k samples. Substituting the median filtered fₙₒᵣₘ(t) for (9) in (10), tissue amplitude modulation can be removed while blood amplitude modulation is preserved,

$$\begin{matrix}{s}_{tissue + blood} ( t ) = ( {\sum }_{m = 0}^{M - 1} {A}_{m} ( t ) + {\sum }_{n = 0}^{N - 1} {A}_{n} ( t ) {e}^{j {ω}_{n} ( t ) t} ) {f}_{norm} ( t ) \\ \approx {\sum }_{m = 0}^{M - 1} {A}_{m} ( t ) + {\sum }_{n = 0}^{N - 1} {A}_{n} ( t ) {e}^{j {ω}_{n} ( t ) t}\end{matrix} \qquad ( 12 )$$

One is then left with (3), which, again, can easily be addressed with conventional filters.

Adaptive Clutter Demodulation Implementation

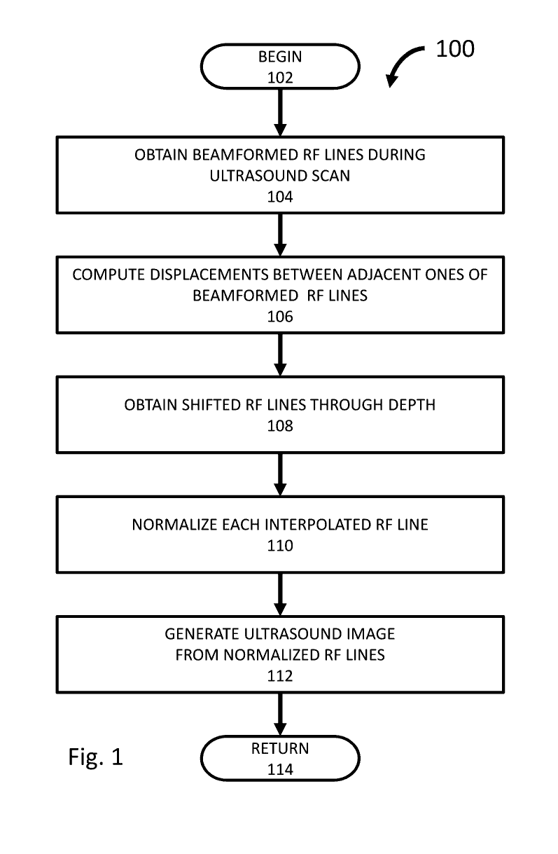

An ultrasound imaging process 100, implementing the described phase demodulation, is shown in FIG. 1. This process can be implemented for processing acquired ultrasound data using software, dedicated hardware, or a combination of software and hardware. In particular embodiments, the process can be implemented by Doppler processor configured to receive the echo data and to output flow data, which can be subsequently combined with anatomic data generated by a conventional ultrasound processor (e.g., a non-Doppler processor) to form a complete image with anatomic and flow data.

Although the methodology described below and discussed above refers primarily to power Doppler methods, the various embodiments are not limited in this regard. Rather, the same type of processing can be used with other ultrasound flow detection methods.

The process begins at step 102 and proceeds to step 104. At step 104, RF lines generated during an ultrasound scan (or rather the data representing such RF lines) is obtained. For example, signals representing echo data received by a transducer of an ultrasound device can be converted to beamformed RF line data. The generation of such data is not limited to any particular method.

Thereafter, at step 106, the displacements between adjacent ones of the beamformed RF lines are obtained. In some embodiments, step 106 can first involve using a standard 2D autocorrelation method to compute relative displacements between temporally adjacent beamformed radio frequency (RF) lines [17]. However, in other embodiments, the displacements can be obtained via other methods, including, but not limited to frequency domain methods, time-domain methods, Bayesian methods, and hybrid approaches. Step 106 can also involve, similar to approaches used in phase aberration estimation [18], [19], computing absolute displacements relative to the first RF line. In some embodiments, this can be performed via some type of modeling technique or approach. For example, absolute displacements can be reconstructed by solving the system of equations in a least square error sense. An exemplary system of equations is shown below in (13):

$$[ \begin{matrix}0 \\ {d}_{01} \\ {d}_{10} \\ {d}_{12} \\ ⋮ \\ {d}_{L - 1 L - 2}\end{matrix} ] = [ \begin{matrix}1 & 0 & 0 & … & 0 \\ - 1 & 1 & 0 & … & 0 \\ 1 & - 1 & 0 & … & 0 \\ 0 & - 1 & 1 & … & 0 \\ ⋮ & ⋮ & ⋮ & ⋱ & ⋮ \\ 0 & 0 & 0 & … & 1\end{matrix} ] [ \begin{matrix}{D}_{0} = 0 \\ {D}_{1} \\ {D}_{2} \\ {D}_{3} \\ ⋮ \\ {D}_{L - 1}\end{matrix} ] \qquad ( 13 )$$

where dₘₙ represents the relative displacement estimate between time steps m and n (assuming that dₘₙ=−dₙₘ, which is not true for all motion estimators), Dₗ represents absolute displacement at slow-time point l, and L represents the number of samples through slow-time.

Once the displacements are computed at step 106, the process can proceed to step 108 where the RF lines are shifted through depth. This step involves performing a non-rigid shift of the RF lines. In some embodiments, this can be implemented via some type of interpolation. For example, step 106 can involve using a shape-preserving piecewise cubic interpolation (or any other interpolation technique) to interpolate each RF line through depth from the computed absolute displacement to zero displacement. This is equivalent to adaptively demodulating the slow-time Doppler signal based on the tissue motion as discussed above with respect to (5). For this method to work, one assumes that the tissue is sufficiently bright relative to the blood signal so that only tissue displacement is reflected in the 2D autocorrelation [20]. However, where the tissue is not sufficiently bright relative to the blood signal, other displacement estimators besides autocorrelation methods can be used. For example, biased motion estimators can be used, such as those that utilize Bayes theorem.

As described in the previous section, to minimize tissue signal amplitude variation through slow-time while still preserving the power of the signal, each shifted RF signal through slow-time is normalized. Thus, at step 110, the shifted RF lines obtained at step 108 can be normalized. For example, the shifted RF lines can be normalized to the amplitude of the envelope of the signal divided by the amplitude of the power of the envelope of the signal. This normalization function is described above in equation (11). In some embodiments, the normalization function is resampled, to account for the non-rigid shift of the RF lines, before the correction (i.e., the normalization) is provided at step 110 to avoid blood signal amplitude demodulation, as described in the previous section. For example, this can involve the normalization function being median filtered through slow-time.

The normalized RF lines can then be used at step 112 to generate an ultrasound image. Step 112 can involve any conventional ultrasound imaging processes, including, but not limited to application of a clutter filter to the normalized RF lines. For example, a traditional infinite impulse filter (IIR) or a spatiotemporal filter based on singular value decomposition (SVD) or a principal component analysis (PCA) type of decomposition. Thereafter, the process can proceed to step 114 to resume previous processing, including repeating process 100 for other ultrasound datasets.

### Examples

The examples shown here are not intended to limit the various embodiments. Rather they are presented solely for illustrative purposes.

Data Acquisition and Beamforming

Channel data from plane wave transmit sequences were acquired using a Verasonics Vantage Ultrasound System (Verasonics, Inc., Kirkland, Wash.), L12-5 linear array probe, and C5-2 curvilinear array probe. Data were acquired at center frequencies of 7.8 MHz and 3.1 MHz to end depths of 3 cm and 8 cm with the L12-5 and C5-2 probes, respectively.

Three different plane wave acquisition methods were used and will be referred to as follows: single plane wave (SPW), plane wave synthetic aperture (PWSA), and multple plane wave (MPW). For the SPW method, 0° plane waves were acquired at a PRF of 1 kHz. For the PWSA case, plane waves angled between −8° to 8° spaced by 2° were acquired at a PRF of 9 kHz. All 9 angles were used to generate a single frame using the method by Montaldo et al., resulting in a final PRF of 1 kHz [10]. For the MPW case, 0° plane waves were acquired at a PRF of 9 kHz and 9 consecutive plane waves were summed together after beamforming to generate a single frame, again resulting in a final PRF of 1 kHz.

All data sets were beamformed with a Harm apodization on receive, and aperture growth with an F/# of 2 was implemented during beamforming. To obtain a final sampling frequency greater than or equal to 50 MHz, RF data were upsampled through depth by an integer number of samples (i.e. 62.5 MHz and 50 MHz for the data acquired with the L12-5 and C5-2 probes, respectively). Before further processing, a FIR band-pass filter was applied to the beamformed RF data.

All beamforming and signal processing for all studies were done in MATLAB R2014a (The MathWorks, Inc., Natick, Mass.).

Sonographer Hand Motion Phantom Experiment

Six volunteers were recruited for three separate trials to acquire channel data of a quality assurance phantom (CIRS Model 040GSE, Norfolk, Va.) for 3 s using a transmit voltage of 30.7V. For the first trial, the L12-5 probe and PWSA acquisition method were used. The 0° plane wave acquisitions from this sequence were used to generate SPW data. For the second trial, the L12-5 probe and MPW acquisition method were used. Since the PWSA and MPW methods theoretically improve SNR, trials 1 and 2 were performed to assess signal-to-noise ratio (SNR) limitations of the standard SPW method that is used for all other studies in this paper. Additionally, since the PWSA method focuses on transmit while the SPW and MPW methods do not, the effects of transmit beamforming induced spectral broadening were also examined. The third trial used the C5-2 probe and SPW acquisition method. This trial provided an assessment of the algorithm at a lower imaging frequency (3.1 MHz), compared to the standard 7.8 MHz imaging frequency used for the rest of the studies. For all three trials, the phantom was stationary, so that sonographer hand motion was the only variable causing clutter motion in the acquired data. All four imaging cases are summarized in Table I and labeled as cases 1 through 4.

For each imaging case, the center line of each plane wave acquisition was beamformed to generate an M-mode image. The adaptive clutter demodulation scheme was performed on each M-mode image. A median filter of 35 samples (35 ms) was used for the amplitude demodulation. Using the same cutoffs as the first band-pass filter, an additional FIR band-pass filter was applied to the RF data through depth. Median full-width bandwidths through depth were computed for dB values down to −100 dB for spectra before and after adaptive demodulation. Median bandwidths were then averaged across subjects. Corresponding velocity estimates in mm/s were computed using

$$v = \frac{f * c * 1000}{2 * {f}_{0}} ,$$

where f is slow-time frequency in Hz, c is the speed of sound in m/s, and f₀ is the center frequency in MHz.

Additionally, to compare the individual effects of the phase and amplitude demodulation steps, bandwidth values were also computed for data without the amplitude demodulation for the SPW case from trial 1 (imaging case 2). Furthermore, two additional median filters of size 71 and 141 samples (71 ms and 141 ms) as well as amplitude demodulation with no median filtering were compared for this case to assess the effects of median filtering on the tissue clutter bandwidth.

No Motion Phantom Experiment

To illustrate inherent scanner amplitude variability and to demonstrate the need for the described amplitude demodulation step in the proposed algorithm, a no motion phantom experiment was performed. The L12-5 probe and SPW acquisition method were used to acquire data of the same quality assurance phantom used for the sonographer hand motion experiment using a transmit voltage of 30.7V. For this experiment, a ring stand and probe holder were used to ensure that no sonographer hand motion, patient physiological motion, or blood flow were present. This imaging case is summarized in Table I and labeled as case 5.

The center line of each plane wave acquisition was beamformed to generate an M-mode RF image. Amplitude through slow-time was qualitatively compared between raw RF data, phase demodulated RF data, and phase and amplitude demodulated (with no median filtering) RF data for two example depths. Additionally, the power of each signal was computed and compared to ensure that the amplitude demodulation preserves signal power as expected.

In Vivo Experiments

Two experiments were performed to assess in vivo feasibility: an arterial occlusion (reactive hyperemia) experiment and a muscle contraction (exercise hyperemia) experiment. Informed written consent in accordance with Vanderbilt University's institutional review board (IRB) was given by two subjects prior to the start of the studies. The first subject was a healthy 35 year old male, and the second subject was a healthy 44 year old male. For both studies, data were acquired at a transmit voltage of 16.1V using the L12-5 probe and SPW method.

For the arterial occlusion experiment, ultrasound data were acquired of the first subject's left gastrocnemius muscle. To prevent the muscle from being compressed against the scanning bed, the subject's left calf was raised slightly and his left foot was secured while lying supine. To ensure continual contact of the probe, hand-held assistance was used in combination with a stationary holder to hold the probe beneath the gastrocnemius muscle. Just above the subject's left knee, a thigh blood pressure cuff (Model CC22, Hokanson, Bellevue, Wash.) was placed and inflated within 1 s to 300 mmHg using a rapid cuff inflator (Model E20, Hokanson, Bellevue, Wash.). To induce arterial occlusion in the calf, the cuff was kept inflated for 5 min [21-23]. Data were acquired for 30 s after the 5 minute occlusion. The cuff was rapidly released about 4 s into the scan. This imaging case is summarized in Table I and labeled as case 6.

For the muscle contraction experiment, ultrasound data were acquired of the second subject's left tibialis anterior muscle. While lying supine, the subject's left calf was slightly raised and his left foot was secured in a custom-built foot device [24]. A trained sonographer held the probe on the tibialis anterior muscle and data were acquired for 30 s. About 8 s into the scan the subject was instructed to dorsiflex his left foot to contract and induce perfusion in the tibialis anterior muscle. After 5 s the subject relaxed his foot. This imaging case is summarized in Table I and labeled as case 7.

For both in vivo studies, for every 50 ms time point, data were broken up into 2 s time frames, and each time point was processed separately. Using parallel receive beamforming, full images were formed from each plane-wave transmit.

Adaptive clutter demodulation was applied to each in vivo time point. A median filter of 35 samples (35 ms) was used for the amplitude demodulation. Using the same cutoffs as the first band-pass filter, an additional FIR band-pass filter was applied to the RF data through depth. Three fourth order Butterworth filtering cases were compared: a 20 Hz high pass applied to adaptively demodulated RF data (proposed filter case), a 20 Hz high pass applied to normal RF data, and a 50 Hz high pass applied to normal RF data. For each method and signal through slow-time, a mirror reflection of the first 20 points was added to the beginning of the signal before filtering and removed after filtering. Power Doppler images were generated by computing amplitude at each pixel using

$$P = \sqrt{\frac{{\sum }_{l = 1}^{L} {s ( l )}^{2}}{L}} ,$$

where L is the number of slow-time points and s(l) is the slow-time signal at time l. A 5×5 spatial median filter and a 7×1 slow-time median filter were applied to each power Doppler image.

For each filtering case and time point, the relative change in power from the last time point was measured at each pixel within a muscle region of interest (ROI). To improve robustness to outliers, for each filtering case, each image was scaled to the 90th percentile of the image with the highest median ROI value. Additionally, due to large motion artifacts during the cuff release and contraction of the muscle, time points with 5th percentile normalized cross correlation values below 0.995 were not included when measuring the dynamic ranges of the relative median power and when scaling the images.

Adaptive demodulation with no median filtering and with median filters of size 71 and 141 samples (71 ms and 141 ms) were also compared for single time point for each in vivo case to further assess the effects of amplitude demodulation.

TABLE 1
Imaging cases are summarized by field of view (FOV), tissue
clutter source, probe, acquisition method, and transmit voltage.

| | | Motion | | | Transmit |
|---|---|---|---|---|---|
| | | Clutter | Probe | Acq. | Voltage |
| Case | FOV | Source | (Frequency) | Method | (V) |
| 1 | Phantom | Hand | L12-5 (7.8 MHz) | PWSA | 30.7 |
| 2 | Phantom | Hand | L12-5 (7.8 MHz) | SPW | 30.7 |
| 3 | Phantom | Hand | L12-5 (7.8 MHz) | MPW | 30.7 |
| 4 | Phantom | Hand | C5-2 (3.1 MHz) | SPW | 30.7 |
| 5 | Phantom | None | L12-5 (7.8 MHz) | SPW | 30.7 |
| 6 | in vivo | Patient & | L12-5 (7.8 MHz) | SPW | 16.1 |
| | | Hand | | | |
| 7 | in vivo | Patient & | L12-5 (7.8 MHz) | SPW | 16.1 |
| | | Hand | | | |

SNR Comparison

The phantom and in vivo studies could potentially result in different SNRs due to different transmit voltages used for the acquisitions. Additionally, the PWSA and MPW methods will likely result in increased SNR. To quantify potential differences in SNR and its effect on the proposed algorithm, SNR was computed in each case using

$$SNR = \frac{ρ}{1 - ρ} ,$$

where ρ is the slow-time RF A-line to A-line normalized cross correlation value [25]. Kernel sizes of 5 and 1.25 wavelengths were used for the normalized cross correlation estimates for the in vivo contraction study and all other studies, respectively. The RF-lines were upsampled to a sampling frequency of 156 MHz to improve the quality of the estimate. A sliding window of 1 sample was used to estimate ρ for every pair of RF lines over the first 2 s of data from the in vivo and phantom acquisitions. A Fisher transformation was performed, and then averaged the estimates of ρ. The SNR was then estimated after performing the inverse transformation.

Results

Sonographer Hand Motion Phantom Results

For the data acquired with the L12-5 probe (7.8 MHz imaging frequency, imaging cases 1-3), adaptive demodulation resulted in median full-width bandwidths through depth averaged across subjects below 20 Hz at −60 dB for all three acquisition methods, allowing for velocities below 1 mm/s to potentially be detected, as seen in FIG. 1a and Table II.

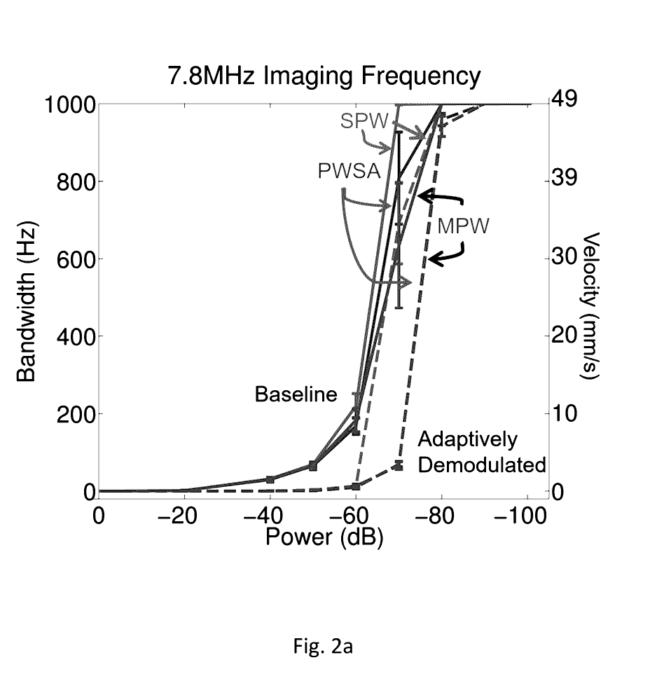

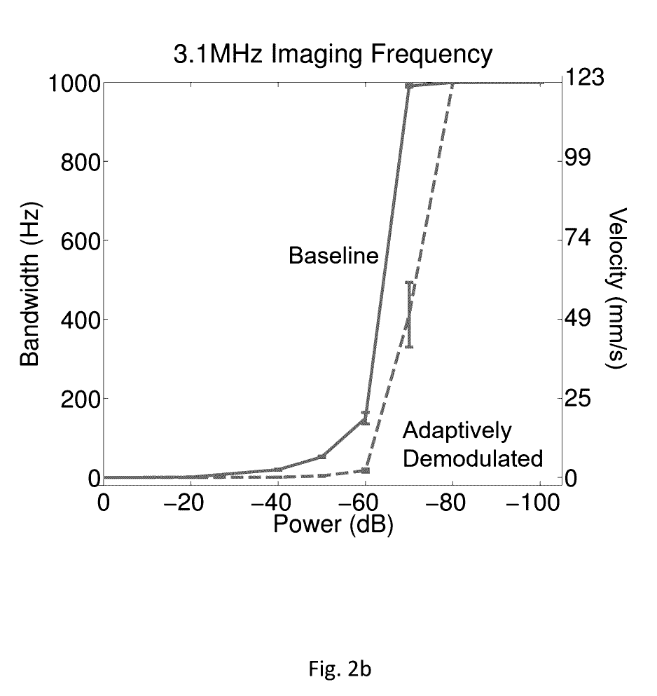

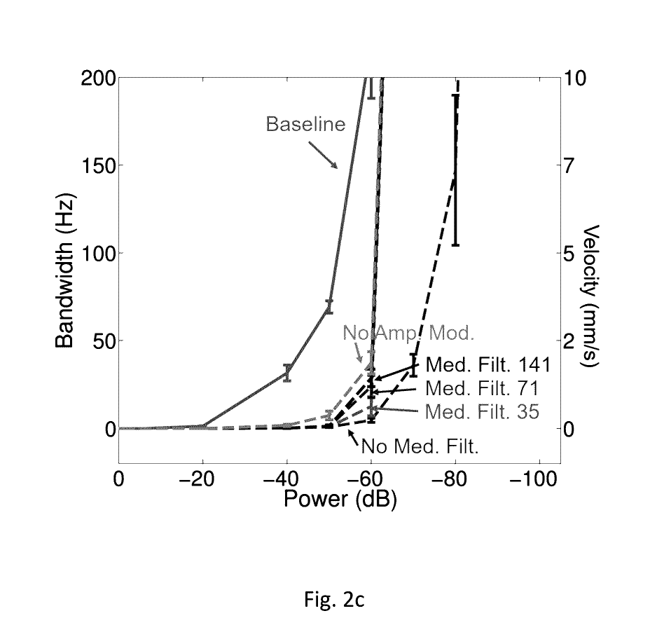

FIG. 2a shows the median bandwidths and corresponding velocities computed across depths and averaged across subjects are shown for three beamforming methods, SPW (teal), PWSA (orange), and MPW (purple), before (solid) and after (dashed) adaptive demodulation (with a 35 sample median filter during amplitude demodulation) at dB values down to −100 dB for the data acquired with the L12-5 probe (7.8 MHz imaging frequency, imaging cases 1-3).

TABLE 2
Median bandwidths (BWs) through depth averaged across subjects
and corresponding velocities at −60 dB before and after adaptive
demodulation for the sonographer hand motion experiment (imaging
cases 1-4). Standard error of the mean is shown in parenthesis for
each measurement.

| Imaging | BW | Velocity Before | BW | Velocity After |
|---|---|---|---|---|
| Case | Before (Hz) | (mm/s) | After (Hz) | (mm/s) |
| 1 | 168 (±21.8) | 8.28 (±1.07) | 9.67 (±4.42) | 0.48 (±0.22) |
| 2 | 220 (±32.0) | 10.8 (±1.58) | 12.6 (±5.40) | 0.62 (±0.27) |
| 3 | 183 (±29.7) | 9.02 (±1.46) | 13.0 (±5.28) | 0.64 (±0.26) |
| 4 | 150 (±14.3) | 18.5 (±1.76) | 17.4 (±4.13) | 2.14 (±0.51) |

For the data acquired with the C5-2 probe (3.1 MHz frequency, imaging case 4), adaptive demodulation resulted in an average median full-width bandwidth of 17.4 Hz at −60 dB, allowing for velocities below 2.14 mm/s to potentially be detected, as shown in FIG. 1b and Table II.

FIG. 2b shows the median bandwidths and corresponding velocities computed across depths and averaged across subjects are shown for the SPW beamforming method before (solid) and after (dashed) adaptive demodulation (with a 35 sample median filter during amplitude demodulation) at dB values down to −100 dB for the data acquired with the C5-2 probe (3.1 MHz imaging frequency, imaging case 4).

FIG. 2c and Table III show the results for the individual effects of phase and amplitude demodulation as well as the effects of median filtering with the amplitude demodulation on the tissue clutter bandwidth for the L12-5 SPW method (7.8 MHz imaging frequency, imaging case 2).

In particular, FIG. 2c shows the median bandwidths and corresponding velocities computed across depths and averaged across subjects at dB values down to −100 dB for the data acquired with L12-5 probe and SPW acquisition method (7.8 MHz imaging frequency, imaging case 2) for different steps of the adaptive demodulation scheme. The bandwidth axis is cropped to highlight differences at −60 dB. Baseline bandwidths are shown as the solid teal line, bandwidths after phase demodulation are shown as the green dotted line, and bandwidths after phase & amplitude demodulation with different sized median filters are shown as follows: 141 (pink dotted line), 71 (purple dotted), 35 (teal dotted), no median filter (orange dotted).

These results show that the amplitude demodulation improves the bandwidth suppression and that smaller median filter sample sizes result in lower bandwidths. At −60 dB, phase demodulation (no amplitude demodulation) resulted in a full-width bandwidth of 37.3 Hz (1.84 mm/s). Amplitude demodulation further improved the bandwidth suppression, with smaller median filter sizes resulting in increased suppression. Amplitude demodulation with no median filtering decreased the bandwidth to 4.69 Hz (0.23 mm/s) while amplitude demodulation with 35 sample median filtering decreased the bandwidth to 12.6 Hz (0.62 mm/s).

TABLE 3
Median bandwidths (BWs) through depth averaged across subjects and
corresponding velocities at −60 dB after adaptive demodulation with
phase demodulation, phase & amplitude demodulation with varying
median filter sizes, and phase & amplitude demodulation with no
median filtering for data acquired with the L12-5 probe and SPW
acquisition method (7.8 MHz imaging frequency, imaging case 2).
Standard error of the mean is shown in parenthesis for each
measurement.

| | | Velocity After |
|---|---|---|
| Demod. Method | BW After (Hz) | (mm/s) |
| Phase | 37.3 (±6.31) | 1.84 (±0.31) |
| Phase & Amp. (No | 4.69 (±1.19) | 0.23 (±0.06) |
| Med. Filt.) | | |
| Phase & Amp. (Med. | 12.6 (±5.40) | 0.62 (±0.27) |
| Filt. 35) | | |
| Phase & Amp. (Med. | 23.8 (±6.33) | 1.17 (±0.31) |
| Filt. 71) | | |
| Phase & Amp. (Med. | 28.7 (±4.93) | 1.42 (±0.24) |
| Filt. 141) | | |

No Motion Phantom Results

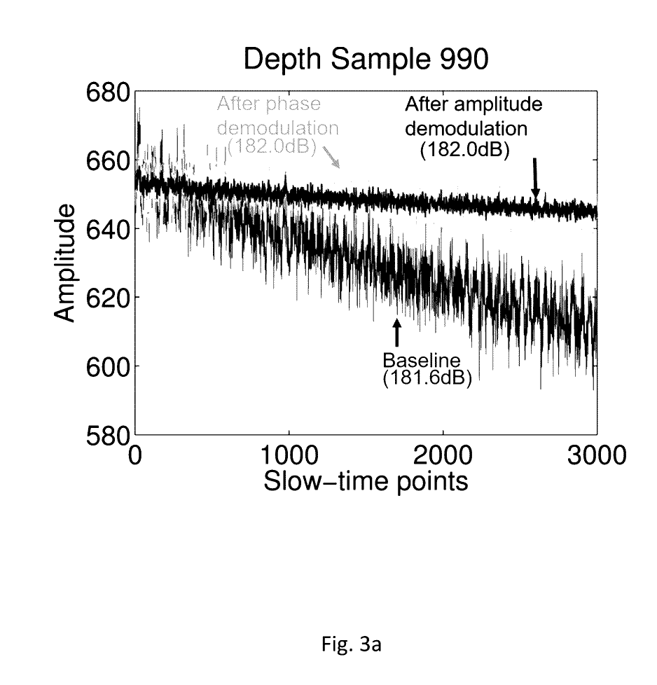

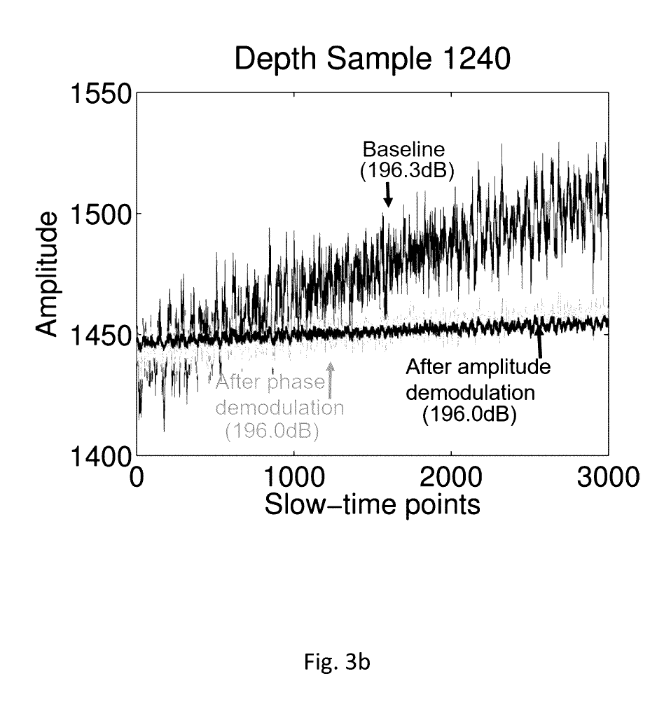

For the no motion phantom case, amplitude at single depths through slow-time are shown in FIGS. 3a and 3b for the baseline RF data, phase demodulated RF data, and phase and amplitude demodulated RF data (without median filtering).

In particular, FIGS. 3a and 3b show amplitude through slow-time for example depths of 990 and 1240. For each depth, amplitude is shown for the raw RF data (medium gray), phase demodulated RF data (light gray), and phase & amplitude demodulated RF data with no median filtering (black). Individual power estimates in dB for each line are shown next to corresponding labels.

Based on these results, since no added motion is present, it is clear that amplitude modulation can result from inherent scanner variation. From the two example depths from the same data set shown in FIGS. 3a and 3b, large variable trends in amplitude modulation are present in the baseline data. Amplitude decreases through slow-time in FIG. 3a while it increases through slow-time in FIG. 3b. For both example depths, phase demodulation is able to correct for this larger bias, while amplitude demodulation is able to correct for additional smaller variations in amplitude, as seen in FIGS. 3a and 3b. Additionally, both phase and amplitude demodulation preserve the power of the baseline signal as seen in the power estimates shown next to corresponding labels in FIGS. 3a and 3b.

In Vivo Results

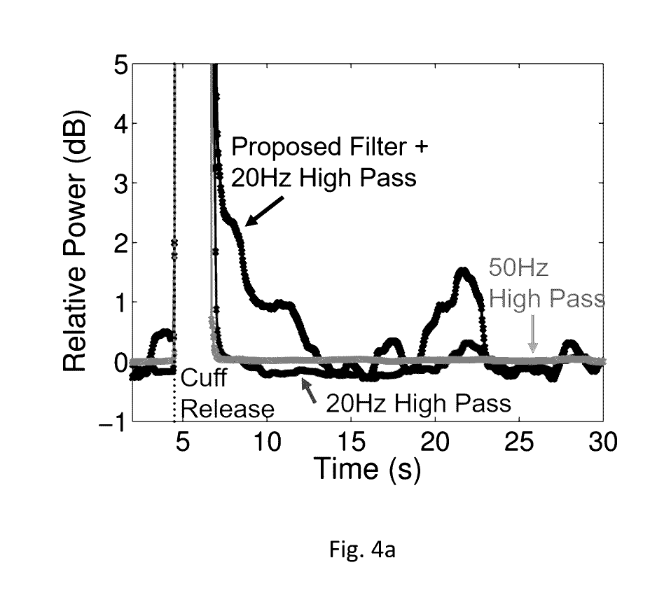

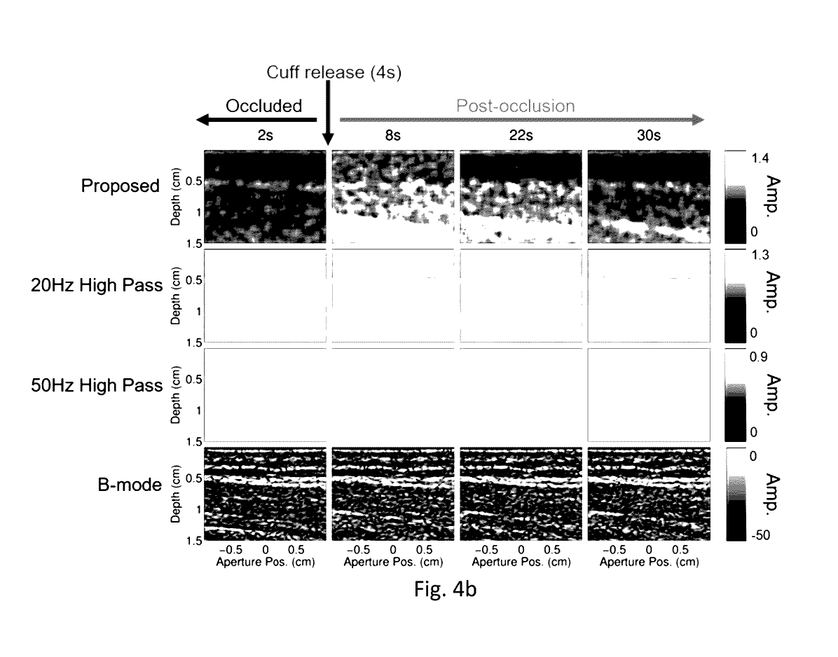

In vivo results are shown in FIGS. 4a, 4b, 5a, 5b, 6a, and 6b.

In particular, FIG. 4a shows the median relative power with respect to the last in vivo arterial occlusion time point is plotted for every 50 ms for each filtering case: proposed filter+20 Hz high pass Butterworth (black), 20 Hz high pass Butterworth (medium gray), and 50 Hz high pass Butterworth (light gray). The time point at which the cuff was released is marked by the dark gray vertical dotted line (at about 4 s). FIG. 4b shows the power Doppler and corresponding B-mode images (bottom row) for 2, 8, 22, and 30 second time points of the in vivo arterial occlusion scan for each filtering case: proposed filter+20 Hz high pass Butterworth (first row), 20 Hz high pass Butterworth (second row), and 50 Hz high pass Butterworth (third row). For each filtering case, each image is scaled to the 90% percentile of the power Doppler image with the highest median value within a fixed muscle ROI. Time points between 4.55 and 6.95 seconds were excluded when computing the scaling for the power Doppler images.

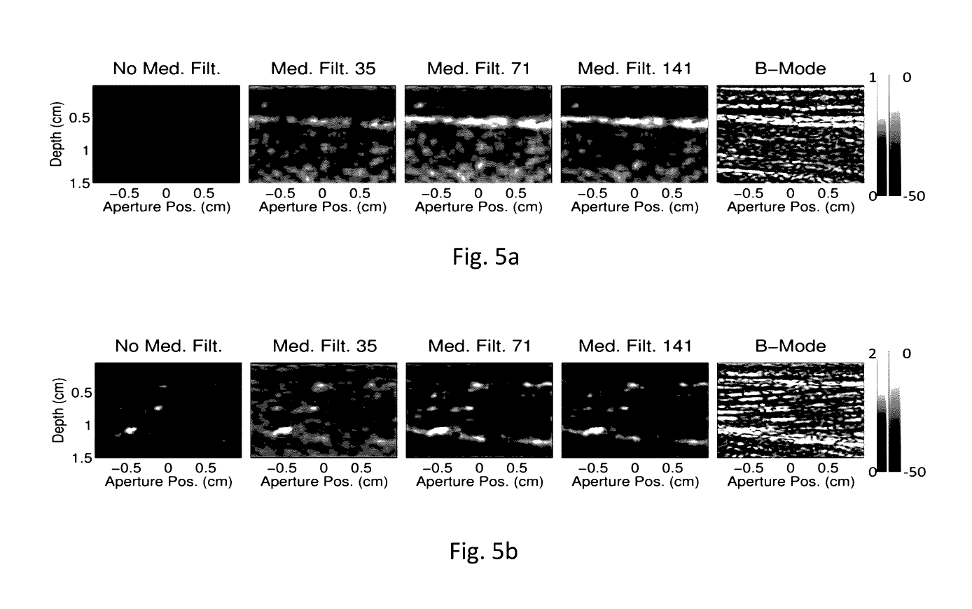

FIGS. 5a and 5b show power Doppler images for the 2 and 5 second time points of the in vivo arterial occlusion scans and muscle contraction scans, respectively, for data processed with no median filter (first column) as well as with median filters of size 35 (second column), 71 (third column), and 141 (fourth column). The corresponding B-mode images are shown in the last column. For each case, the power Doppler images are scaled to the 90% percentile of the power Doppler image with the highest median value within a fixed muscle ROI across all time points. For the arterial occlusion images in (a), time points between 4.55 and 6.95 seconds were excluded when computing the scaling for the power Doppler images. The power Doppler color bar in this figure is rounded to the closest decimal to summarize all three cases, but the actual maximums of each image from left to right are as follows: 1.1, 1.4, 1.3, and 1.4. For the muscle contraction images in (b), time points between 7.7 and 15.65 seconds were excluded when computing the scaling for the power Doppler images. The power Doppler color bar in this figure is rounded to the closest decimal to summarize all three cases, but the actual maximums of each image from left to right are as follows: 1.3, 1.6, 1.8, and 1.9.

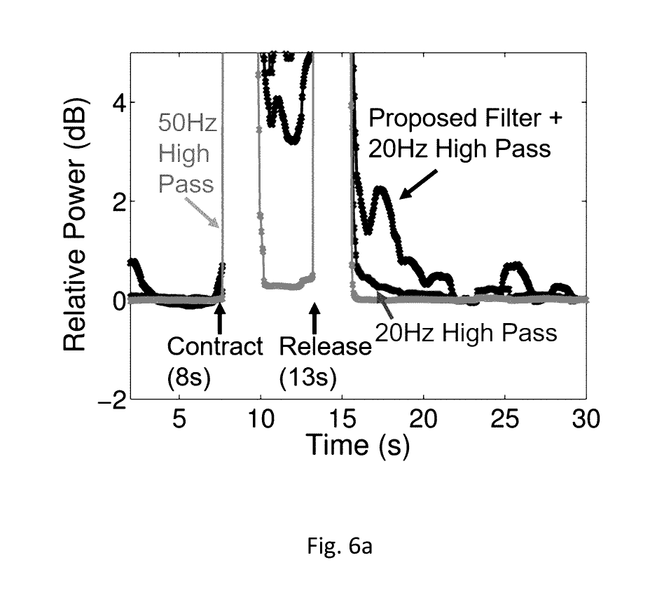

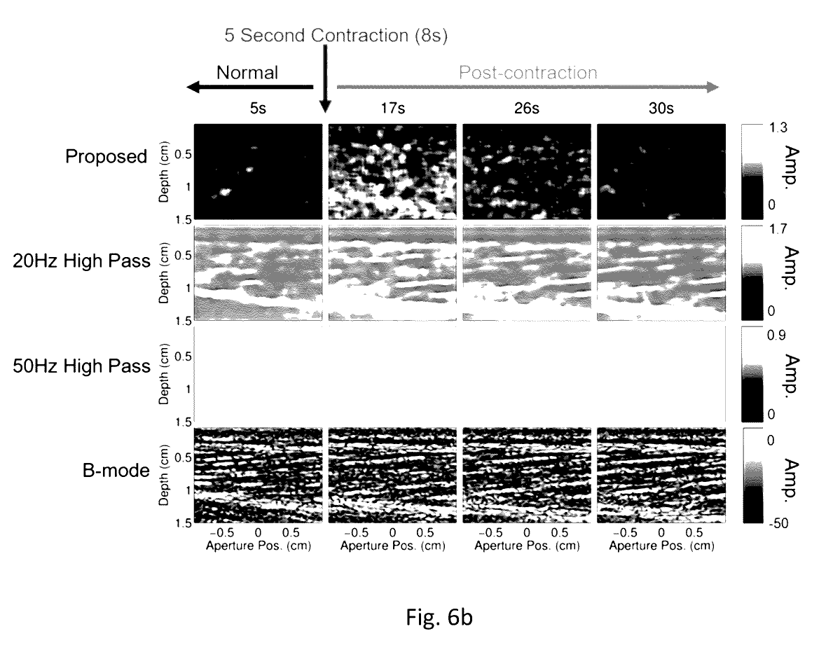

FIG. 6a shows the median relative power with respect to the last in vivo muscle contraction time point is plotted for every 50 ms for each filtering case: proposed filter+20 Hz high pass Butterworth (black), 20 Hz high pass Butterworth (medium gray), and 50 Hz high pass Butterworth (light gray). The time points at which the muscle contracted and released are marked with arrows (at about 8 s and 13 s, respectively). FIG. 6b shows power Doppler and corresponding B-mode images (bottom row) are shown for 5, 17, 26, and 30 second time points of the in vivo muscle contraction scan for each filtering case: proposed filter+20 Hz high pass Butterworth (first row), 20 Hz high pass Butterworth (second row), and 50 Hz high pass Butterworth (third row). For each filtering case, each image is scaled to the 90% percentile of the power Doppler image with the highest median value within a fixed muscle ROI. Time points between 7.7 and 15.65 seconds were excluded when computing the scaling for the power Doppler images.

Turning first to FIGS. 4a and 4b, FIG. 4a shows that the proposed filter case resulted in a larger dynamic range compared to the two conventional filters for the in vivo arterial occlusion study. Dynamic ranges between the highest and lowest blood flow time points were 4.73 dB, 2.1 dB and 0.15 dB for the proposed, 20 Hz conventional, and 50 Hz conventional filters, respectively. FIG. 4b further supports these results qualitatively. Compared to the conventional filter cases, the proposed filter case shows larger differences between the time point during occlusion (2 s) and the time points after occlusion (8, 22, and 30 s). Additionally, the conventional filter cases show structure that is strongly correlated to structure seen in the B-mode images, whereas the proposed filter case exhibits more independent structure, especially within the muscle ROI (between 0.5 and 1 cm depths).

Time points between 4.55 s and 6.95 s were excluded when determining the axis in FIG. 4a and the scaling of the images in FIG. 4b. Excluded time points had 5th percentile normalized cross correlation values below 0.995.

As noted above, FIG. 5a compares power Doppler images at the 2 second time point of different median filter sizes used during amplitude demodulation. Increasing the size of the median filter increases the amount of B-mode structure seen in the power Doppler image. This trend supports the results from the hand motion study which showed increased suppression of the tissue clutter with decreased median filter sizes used for the amplitude demodulation.

Similar results were observed for the muscle contraction study. FIG. 6a shows that the proposed filter case resulted in a larger dynamic range compared to the two conventional filters for the in vivo muscle contraction study. Dynamic ranges between the highest and lowest blood flow time points were 4.80 dB, 1.95 dB and 0.16 dB for the proposed, 20 Hz conventional, and 50 Hz conventional filters, respectively.

FIG. 6b further supports these results qualitatively. Compared to the conventional filter cases, the proposed filter case shows larger differences between the time point before contraction (5 s) and the time points after contraction (17 and 26 s). Again, the conventional filter cases show structure that is strongly correlated to structure seen in the B-mode images, whereas the proposed filter case exhibits more independent structure, especially within the muscle ROI (between 0.5 and 1 cm depths).

Time points between 7.7 s and 15.65 s were excluded when determining the axis in FIG. 6a and the scaling of the images in FIG. 6b. Excluded time points had 5th percentile normalized cross correlation values below 0.995.

FIG. 5b shows power Doppler images at the 5 second time point for the proposed filter case with different median filter sizes used for the amplitude demodulation. Similar to the in vivo occlusion study, increasing the size of the median filter appears to increase the amount of B-mode structure seen in the power Doppler image. Again, this trend supports the results from the hand motion study which showed increased suppression of the tissue clutter with decreased median filter sizes used for the amplitude demodulation.

SNR Results

SNR values for the hand motion data acquisitions were computed to be 37.7 dB, 45.3 dB, and 46.4 dB, for the SPW, PWSA, and MPW sequences, respectively. For the in vivo data, SNR was computed to be 36.8 dB and 40.9 dB, for the occlusion and contraction studies, respectively.

### Discussion

The sonographer hand motion bandwidth results demonstrate that adaptive demodulation according to the various embodiments can isolate slow flow velocities from tissue clutter at relative blood signals at least 60 dB lower than the tissue. Others have reported the amplitudes of blood signals as being up to 100 dB lower than tissue clutter [3]. For the tested system, the signal to noise limit is reached around 70 dB below tissue after adaptive demodulation. The PWSA and MPW sequence modifications used for the hand motion studies both improve SNR and lower the noise floor by about 10 dB (as seen in FIG. 1a). However, the MPW case did not result in improved bandwidth suppression at −60 dB, and only a minor improvement with the PWSA sequence was observed (as seen in Table II). Since the PWSA sequence also improves resolution and image quality, the improvement seen with PWSA, although small, is not negligible and is likely due to decreased intrinsic spectral broadening resulting from the transmit beam shape. Since the SPW and MPW methods have lower resolution due to unfocused transmit beams, intrinsic spectral broadening will result from geometrical focal broadening during receive beamforming, resulting in larger bandwidths of the tissue clutter [26]. This suggests that intrinsic spectral broadening is a more immediate limitation of the methods described herein compared to SNR. Apart from sequence modifications, higher transmit voltage also improves SNR. However, the use of a higher transmit voltage (30.7V) for the hand motion phantom SPW study did not result in a substantially higher SNR value compared to the in vivo studies which used the same acquisition sequence using only half the transmit voltage (16.1V). This is likely due to the phantom and in vivo data being acquired on different media. However, this further suggests that SNR is not a significant direct limitation of the method of the various embodiments.

The stationary phantom experiment illustrated the presence of signal amplitude modulation through slow-time even when no blood flow, hand motion, or patient physiological motion are present. The results from this experiment demonstrate that the amplitude demodulation process of the various embodiments can be used to significantly suppress the tissue clutter bandwidth. In principle, the amplitude demodulation method described herein proposed would also affect the blood signal amplitude. Accordingly, as discussed above, median filtering of the amplitude demodulation function can be used to ensure that tissue signal is the primary contributor to the amplitude modulation estimate. Ideally, the median filter should be long enough to avoid suppression of the blood signal and short enough to allow for maximal tissue clutter suppression. For example, if blood flows outside of a given pixel faster than the acquisition rate (i.e. blood is incoherent between consecutive acquisitions), small median filter sizes would suffice. Although little is known about the coherence length of slow flow, it is plausible that when imaging slow flow, the blood signal will be coherent across a longer time than with fast flow. Therefore, larger filter sizes would be required to ensure the blood signal amplitude is not demodulated. A median filter of 35 samples was used for the experiments described herein.

Expected reactive and exercise hyperemia behaviors are well characterized, which provided reasonable standards for an algorithm in accordance with the various embodiments in applicable clinical settings. Both the arterial occlusion and contraction studies are consistent with previous findings of perfusion and blood-oxygen-level-dependent (BOLD) MRI characteristics under these conditions. For the arterial occlusion study, Lebon et al. and Englund et al. both showed time to peak perfusion times between 15 and 20 s after arterial occlusion in the calf muscle, which agrees with the results shown in FIG. 4a [27, 28]. For the adaptively demodulated data with a 20 Hz high pass filter in accordance with an embodiment, the peak blood flow time point occurs at about 22 s, which is about 18 s after the cuff was released (cuff was released at about 4 s), as seen in FIG. 4a. Similarly for the contraction study, Towse et al. showed that peak BOLD signal intensity occurs between 5-7 s post-muscle contraction, which correlates with the results shown in FIG. 6a [24, 29]. In FIG. 6a, the proposed filter method of the various embodiments resulted in peak power at about 17 s which is approximately 5 s post-contraction (last contraction time point occurs at about 12 s). It is also important to note that the median power Doppler values in both FIGS. 4a and 6a are referenced to the last time point for each case. Although power Doppler values below or at least at zero during occlusion and before contraction were expected, assuming there should be increased perfusion post-occlusion and post-contraction, this was not the case. However, results found in the literature show similar trends in perfusion values, with post-occlusion and post-contraction values even decreasing to values below those during occlusion and before contraction after reaching peak perfusion [24, 27, 28].

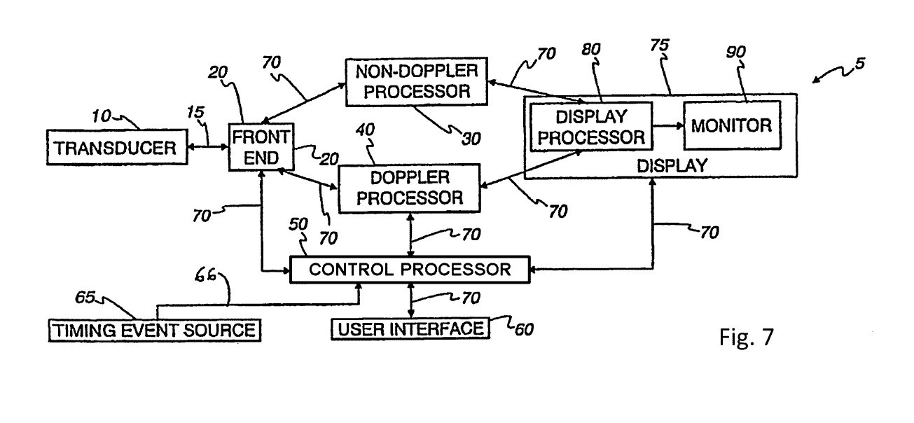

FIG. 7 illustrates an embodiment of an ultrasound machine, generally designated 5, that can be configured for implementing the various embodiments. A transducer 10 (including a probe for example) transmits ultrasound waves into a subject (live tissue for example) by converting electrical analog signals to ultrasonic energy and receives the ultrasound waves backscattered from the subject by converting ultrasonic energy to analog electrical signals. A front-end 20, that in one embodiment includes a receiver, transmitter, and beamformer, may be used to create the necessary transmitted waveforms, beam patterns, receiver filtering techniques, and demodulation schemes that are used for the various imaging modes. Front-end 20 performs such functions, converting digital data to analog data and vice versa. Front-end 20 interfaces to transducer 10 using analog interface 15 and interfaces to a non-Doppler processor 30, a Doppler processor 40 and a control processor 50 over a bus 70 (digital bus for example). Bus 70 may include several digital sub-buses, each sub-bus having its own unique configuration and providing digital data interfaces to various parts of the ultrasound machine 5.

Non-Doppler processor 30 is, in one embodiment, adapted to provide amplitude detection functions and data compression functions (e.g., image compression and formation methods) used for imaging modes such as B-mode, M-mode, and harmonic imaging. Doppler processor 40, in one embodiment provides clutter filtering functions and movement parameter estimation functions used for imaging modes such as tissue velocity imaging (TVI), strain rate imaging (SRI), and color M-mode and B-mode. In one embodiment, the two processors, 30 and 40, accept digital signal data from the front-end 20, process the digital signal data into estimated parameter values, and pass the estimated parameter values to processor 50 and a display 75 over digital bus 70. The estimated parameter values may be created using the received signals in frequency bands centered at the fundamental, harmonics, or sub-harmonics of the transmitted signals in a manner known to those skilled in the art.

Display 75 is adapted, in one embodiment, to provide scan-conversion functions, color mapping functions, and tissue/flow arbitration functions, performed by a display processor 80 which accepts digital parameter values from processors 30, 40, and 50, processes, maps, and formats the digital data for display, converts the digital display data to analog display signals, and communicate the analog display signals to a monitor 90. Monitor 90 accepts the analog display signals from display processor 80 and displays the resultant image.

A user interface 60 enables user commands to be input by the operator to the ultrasound machine 5 through control processor 50. User interface 60 may include a keyboard, mouse, switches, knobs, buttons, track balls, foot pedals, a microphone for inputting voice commands and on-screen menus, among other devices.

A timing event source 65 generates a cardiac timing event signal 66 that represents the cardiac waveform of the subject. The timing event signal 66 is input to ultrasound machine 5 through control processor 50.

In one embodiment, control processor 50 includes the main, central processor of the ultrasound machine 5, interfacing to various other parts of the ultrasound machine 5 through digital bus 70. Control processor 50 executes the various data algorithms and functions for the various imaging and diagnostic modes. Digital data and commands may be communicated between control processor 50 and other various parts of the ultrasound machine 5. As an alternative, the functions performed by control processor 50 may be performed by multiple processors, or may be integrated into processors 30, 40, or 80, or any combination thereof. As a further alternative, the functions of processors 30, 40, 50, and 80 may be integrated into a single PC backend.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments. Rather, the scope of the invention should be defined in accordance with the following claims and their equivalents.

Although the invention has been illustrated and described with respect to one or more implementations, equivalent alterations and modifications will occur to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

### REFERENCES

The following documents, which are referred to throughout and the contents of each of which are incorporated by reference in their entirety, are useful for understanding the various embodiments.
[1] C. Tremblay-Darveau, R. Williams, L. Milot, M. Bruce, and P. N. Burns, “Combined perfusion and doppler imaging using plane-wave nonlinear detection and microbubble contrast agents,” IEEE Transactions on Ultrasonics, Ferroelectrics and Frequency Control, vol. 61, pp. 1988-2000, 2014.
[2] J. A. Jensen, Estimation of Blood Velocities Using Ultrasound: A Signal Processing Approach. Cambridge: Cambridge UP, 1996.
[3] S. Bjaerum, H. Torp, and K. Kristoffersen, “Clutter filters adapted to tissue motion in ultrasound color flow imaging,” IEEE Transactions on Ultrasonics, Ferroelectrics, and Frequency Control, vol. 49, pp. 693-704, 2002.
[4] A. Heimdal and H. Torp, “Ultrasound doppler measurements of low velocity blood flow: limitations due to clutter signals from vibrating muscles,” IEEE Transactions on Ultrasonics, Ferroelectrics and Fre [symbol] quency Control, vol. 44, pp. 873-881, 1997.
[5] S. Bjaerum, H. Torp, and K. Kristoffersen, “Clutter filter design for ultrasound color flow imaging,” IEEE Transactions on Ultrasonics, Ferroelectrics, and Frequency Control, vol. 49, pp. 204-216, 2002.
[6] S. Fadnes, S. Bjaerum, H. Torp, and L. Lovstakken, “Clutter filtering influence on blood velocity estimation using speckle tracking,” IEEE Transactions on Ultrasonics, Ferroelectrics, and Frequency Control, vol. 62, pp. 2079-2091, 2015.
[7] C. Demene, T. Deffieux, M. Pernot, B. F. Osmanski., V. Biran, S. Franqui, and M. Tanter, “Spatiotemporal clutter filtering of ultrafast ultrasound data highly increases doppler and fultrasound sensitivity,” IEEE Transactions on Medical Imaging, vol. 34, pp. 2271-2285, 2015.
[8] G. J. Tangelder, D. W. Slaaf, A. M. Muijtjens, T. Arts, M. G. oude Egbrink, and R. S. Reneman, “Velocity profiles of blood platelets and red blood cells flowing in arterioles of the rabbit mesentery,” Circulation Research, vol. 59, pp. 505-514, 1986.
[9] J. Udesen, F. Gran, K. L. Hansen, J. A. Jensen, C. Thomsen, and M. B. Nielsen, “High frame-rate blood vector velocity imaging using plane waves: Simulations and preliminary experiments,” IEEE Transactions on Ultrasonics, Ferroelectrics, and Frequency Control, vol. 55, pp. 1729-1743, 2008.
[10] G. Montaldo, M. Tanter, J. Bercoff, N. Benech, and M. Fink, “Coherent plane-wave compounding for very high frame rate ultrasonography and transient elastography,” IEEE Transactions on Ultrasonics, Ferro-electrics, and Frequency Control, vol. 56, pp. 489-506, 2009.
[11] L. Thomas and A. Hall, “An improved wall filter for flow imaging of low velocity flow,” IEEE Ultrasonics Symposium Proceedings, vol. 3, pp. 1701-1704, 1994.
[12] C. Gallippi and G. Trahey, “Adaptive clutter filtering via blind source separation for two-dimensional ultrasonic blood velocity measurement,” Ultrasonic Imaging, vol. 24, pp. 193-214, 2002.
[13] M. A. Lediju, G. E. Trahey, B. C. Byram, and J. J. Dahl, “Short-lag spatial coherence of backscattered echoes: Imaging characteristics,” IEEE Transactions on Ultrasonics, Ferroelectrics, and Frequency Con [symbol] trol, vol. 58, pp. 1377-1388, 2011.
[14] Y. L. Li and J. J. Dahl, “Coherent flow power doppler (cfpd): Flow detection using spatial coherence beamforming,” IEEE Transactions on Ultrasonics, Ferroelectrics, and Frequency Control, vol. 62, pp. 1022-1035, 2015.
[15] J. E. Tierney, D. M. Dumont, and B. C. Byram, “Perfusion imaging with non-contrast ultrasound,” in Proc. SPIE9790, Medical Imaging 2016: Ultrasonics Imaging and Tomography, San Diego, Calif., USA, April 2016.
[16] J. E. Tierney, C. Coolbaugh, T. Towse, and B. C. Byram, “Plane wave perfusion ultrasound imaging without contrast,” in Proceedings of the IEEE International Ultrasonics Symposium, Tours, France, September 2016.
[17] T. Loupas, J. T. Powers, and R. W. Gill, “An axial velocity estimator for ultrasound blood flow imaging, based on a full evaluation of the doppler equation by means of a two-dimensional autocorrelation approach,” IEEE Transactions on Ultrasonics, Ferroelectrics, and Frequency Con [symbol] trol, vol. 42, pp. 672-688, 1995.
[18] J. J. Dahl, S. A. McAleavey, G. F. Pinton, M. S. Soo, and G. E. Trahey, “Adaptive imaging on a diagnostic ultrasound scanner at quasi real-time rates,” IEEE Transactions on Ultrasonics, Ferroelectrics, and Frequency Control, vol. 53, pp. 1832-1843, 2006.
[19] R. C. Guass, G. E. Trahey, and M. S. Soo, “Wavefront estimation in the human breast,” in Medical Imaging 2001. International Society for Optics and Photonics, May 2001, pp. 172-181.
[20] G. F. Pinton, J. J. Dahl, and G. E. Trahey, “Rapid tracking of small displacements with ultrasound,” IEEE Transactions on Ultrasonics, Ferroelectrics, and Frequency Control, vol. 53, pp. 1103-1117, 2006.
[21] R. B. Thompson, R. J. Aviles, A. Z. Faranesh, K. Venkatsh, V. Wright, R. S. Balaban, and R. J. Lederman, “Measurement of skeletal muscle perfusion during postischemic reactive hyperemia using contrast-enhanced mri with a step-input function,” Magnetic resonance in medicine, vol. 54, pp. 289-298, 2005.
[22] D. Lopez, A. Pollak, C. H. Meyer, R. Jiji, F. H. Epstein, J. R. Hunter, J. M. Christopher, and C. M. Kramer, “Asl demonstrates higher and more homogenous calf muscle perfusion with post-occlusion hyperemia than with exercise,” Resonance, vol. 15, p. 216, 2013.
[23] T. F. Towse, B. T. Childs, S. A. Sabin, E. C. Bush, C. P. Elder, and B. M. Damon, “Comparison of muscle bold responses to arterial occlusion at 3 and 7 tesla,” Resonance, 2015.
[24] T. F. Towse, J. M. Slade, and R. A. Meyer, “Effect of physical activity on mri-measured blood oxygen level-dependent transients in skeletal muscle after brief contractions,” Journal of Applied Physiology, vol. 99, pp. 715-722, 2005.
[25] B. H. Friemel, L. N. Bohs, K. R. Nightingale, and G. E. Trahey, “Speckle decorrelation due to two-dimensional flow gradients,” IEEE Transactions on Ultrasonics, Ferroelectrics, and Frequency Control, vol. 45, pp. 317-327, 1998.
[26] G. Guidi, C. Licciardello, and S. Falteri, “Intrinsic spectral broadening (isb) in ultrasound doppler as a combination of transit time and local geometrical broadening,” Ultrasound in Medicine and Biology, vol. 26, pp. 853-862, 2000.
[27] V. Lebon, P. G. Carlier, C. Brillault-Salvat, and A. Leroy-Willig, “Si [symbol] multaneous measurement of perfusion and oxygenation changes using a multiple gradient-echo sequence: application to human muscle study,” Magnetic Resonance Imaging, vol. 16, pp. 721-729, 1998.
[28] E. K. Englund, M. C. Langham, C. Li, Z. B. Rodgers, T. F. Floyd, and E. R. Mohler, “Combined measurement of perfusion, venous oxygen saturation, and skeletal muscle t2* during reactive hyperemia in the leg,” Journal of Cardiovascular Magnetic Resonance, vol. 15, pp. 1-13, 2013.
[29] T. F. Towse, J. M. Slade, J. A. Ambrose, M. C. DeLano, and R. A. Meyer, “Quantitative analysis of the postcontractile blood-oxygenation-level-dependent (bold) effect in skeletal muscle,” Journal of Applied Physiology, vol. 111, pp. 27-39, 2011.

What is claimed is:

1. A method, comprising:
obtaining a plurality of beamformed radiofrequency (RF) lines representing an ultrasound scan;
computing displacements between adjacent ones of the plurality of beamformed RF lines;
generating shifted RF lines through depth based on the computed displacements;
normalizing the shifted RF lines to yield normalized RF lines; and
assembling an ultrasound image based on the normalized RF lines.

2. The method of claim 1, wherein the computing comprises:
calculating relative displacements between the plurality of beamformed RF lines; and
reconstructing absolute displacements for the plurality of beamformed RF lines based on the relative displacements.

3. The method of claim 2, wherein the relative displacements are calculated using an autocorrelation method.

4. The method of claim 2, wherein the absolute displacements are reconstructed by a modeling approach.

5. The method of claim 1, wherein the generating of the shifted lines comprises calculating a non-rigid shift of the plurality of beamformed RF lines from the computed absolute displacement to zero displacement.

6. The method of claim 5, wherein the calculating of the non-rigid shift is performed using an interpolation.

7. The method of claim 1, wherein the normalizing comprises scaling the shifted RF lines according to a ratio of a power of an envelope of the plurality of beamformed RF lines and an amplitude of the envelope of the plurality of beamformed RF lines.

8. The method of claim 7, wherein the normalizing further comprises, prior to the scaling, resampling the normalization function to account for non-rigid shifts in the shifted RF lines.

9. A ultrasound system, comprising:
at least one transducer front end;
at least one display processor configured for assembling an ultrasound images based on received RF line data; and
at least one Doppler processor coupled to the at least one transducer front end and the at least one display processor, the at least one Doppler processor configured for:
obtaining, from the at least one transducer front end, a plurality of beamformed RF lines associated with an ultrasound scan,
computing displacements between adjacent ones of the plurality of beamformed RF lines,
generating shifted RF lines through depth based on the computed displacements,
normalizing the shifted RF lines to yield normalized RF lines, and
transmitting data representing the normalized RF lines to the at least one display processor.

10. The ultrasound system of claim 9, wherein the computing comprises:
calculating relative displacements between the plurality of beamformed RF lines; and
reconstructing absolute displacements for the plurality of beamformed RF lines based on the relative displacements.

11. The ultrasound system of claim 10, wherein the relative displacements are calculated using an autocorrelation method.

12. The ultrasound system of claim 10, wherein the absolute displacements are reconstructed by a modeling approach.

13. The ultrasound system of claim 9, wherein the generating of the shifted lines comprises calculating a non-rigid shift of the plurality of beamformed RF lines from the computed absolute displacement to zero displacement.

14. The ultrasound system of claim 13, wherein the calculating of the non-rigid shift is performed using an interpolation.

15. The ultrasound system of claim 9, wherein the normalizing comprises scaling the shifted RF lines according to a ratio of a power of an envelope of the plurality of beamformed RF lines and an amplitude of the envelope of the plurality of beamformed RF lines.

16. The ultrasound system of claim 15, wherein the normalizing further comprises, prior to the scaling, resampling the normalization function to account for non-rigid shifts in the shifted RF lines.

17. A non-transitory computer-readable medium, having stored thereon a computer program executable by a computing device, computer program comprising a plurality of code sections for causing the computing device to:
obtain a plurality of beamformed radiofrequency (RF) lines representing an ultrasound scan;
compute displacements between adjacent ones of the plurality of beamformed RF lines;
generate shifted RF lines through depth based on the computed displacements;
normalize the shifted RF lines to yield normalized RF lines; and
assemble an ultrasound image based on the normalized RF lines.

18. The computer-readable medium of claim 17, wherein the plurality of code sections for the computing further comprise code sections for causing the computing device to:
calculate relative displacements between the plurality of beamformed RF lines; and
reconstruct absolute displacements for the plurality of beamformed RF lines based on the relative displacements.

19. The computer-readable medium of claim 17, wherein the plurality of code sections for the generating of the shifted lines comprises code sections for causing the computing device to calculate a non-rigid shift of the plurality of beamformed RF lines from the computed absolute displacement to zero displacement.

20. The computer-readable medium of claim 17, wherein the plurality of code sections for the normalizing comprises code sections for causing the computing device to scale the shifted RF lines according to a ratio of a power of an envelope of the plurality of beamformed RF lines and an amplitude of the envelope of the plurality of beamformed RF lines.
